(19) United States
(12) Patent Application Publication
(10) Pub. No.: US 2024/0156618 A1
(43) Pub. Date: May 16, 2024

(54) Facet Fusion Tube Assembly

(71) Applicant: William Aaron Kunkle, Fort Wayne, IN (US)

(72) Inventor: William Aaron Kunkle, Fort Wayne, IN (US)

(21) Appl. No.: 18/419,091

(22) Filed: Jan. 22, 2024

Related U.S. Application Data
(63) Continuation of application No. 17/450,715, filed on Oct. 13, 2021, now Pat. No. 11,877,936.
(60) Provisional application No. 63/113,229, filed on Nov. 13, 2020.

(51) Int. Cl. A61F 2/46; A61F 2/44

(52) U.S. Cl. CPC A61F 2/4611; A61F 2/4405; A61F 2/4455; A61F 2002/30069; A61F 2002/4687

(57) ABSTRACT

A facet fusion tube assembly includes an elongated tube that defines a working channel to accept tools, instruments or materials for conducting a facet fusion. The tube includes a clip on its outer circumference that is configured to engage the outer surface of an adjacent tool or instrument used to access and engage the pedicle. A centering cap can be mounted at the proximal end of the elongated tube to align and center a working tool, such as a burring tool. An inner removable rod can extend through the tube to prevent soft tissue migration into the tube during insertion, and can be used for bone graft packing once the facet joint or other area of the spine is prepared for fusion. In use, the tube is clipped onto an adjacent instrument or tool to anchor the facet fusion tube assembly in a proper orientation relative to the facet joint.

### PRIORITY CLAIM

This application is a continuation application of U.S. Ser. No. 17/450,715, filed on Oct. 13, 2021, filing from and claiming priority to U.S. Provisional Application No. 63/113,229, filed on Nov. 13, 2020. The entire contents of the above-mentioned patent applications are incorporated herein by reference as part of the disclosure of this U.S. application.

### BACKGROUND

The present invention relates to the field of minimally invasive spine surgery and more particularly to instruments and methods for reliable and enhanced access to the facet joints.

There are numerous devices to allow for minimally invasive access to the posterior spine. Examples of devices designed for minimal access to the posterior spine are disclosed in U.S. Pat. No. 7,226,451 ('451 Patent), issued on Jun. 5, 2007, and U.S. Pat. No. 7,736,305 ('305 Patent), issued on Jun. 15, 2010. The devices disclosed in these patents are geared toward access for pedicle screw insertion, for rod or plate connection with the pedicle screws, and for access and insertion of biomechanical interbody implants for fusion. These devices do not specifically address the requirements for facet fusion or provide a specific technique or device for facet fusion. U.S. Pat. No. 7,935,136 ('136 Patent), which issued on May 3, 2011, describes a specific type of tether to wrap around the superior and inferior articular process that form a facet joint, as well as an aid in fusion.

The devices in '451 and '305 Patents utilize a minimally invasive tube to access the posterior spine, but the purpose of these devices is to allow for easier access to pedicle screws, rod insertion and assembly and access to the interbody space to allow of insertion of an interbody fusion device. The device in the '136 Patent attempts to address a technique to enhance facet fusion with a tether device but does not do it in a minimally invasive way. The device of the '136 Patent still requires open surgical techniques that necessarily require stripping of significant paraspinal muscle, which can potentially lead to delayed surgical recovery, muscle necrosis, increased blood loss and morbidity.

U.S. application Ser. No. 2013/0253591 ('591 Publication), published on September 26, 2013, discloses a device designed for minimally invasive spine fusion, in particular a longitudinally-directed tube in a cephalad to caudal orientation with split in the tube. The tube in the '591 Publication does not allow for complete decortication or access to the facet joint because it is not configured to allow access for a high-speed burr or drill directly (in a posterior to anterior orientation) onto the facet joint. The device in '591 Publication does not ensure reliable access to the facet joint via direct association to a pedicle cannulation device. In addition, this device targets a posterolateral area for spine fusion with decortication of the transverse process.

There is a need for an apparatus and techniques that overcome the disadvantages of the prior systems discussed above.

### SUMMARY OF THE DISCLOSURE

A facet fusion tube assembly includes an elongated tube of a length sufficient to provide access to the facet joint of any vertebral level. The tube is hollow and open at both ends to operate as a working channel to accept tools, instruments or materials for conducting a facet fusion. The tube includes a clip that is mounted to the outer circumference of the tube and that is configured to engage the outer surface of an adjacent tool or instrument, particularly tools or instruments used to access and engage the pedicle. In one feature, the tube assembly is provided with a centering cap that can be mounted at the distal end of the elongated tube to align and center a working tool, such as a burring tool. In another feature, an inner removable rod extends through the tube to prevent soft tissue migration into the tube during insertion, and to be used for bone graft packing once the facet joint or other area of the spine is prepared for fusion.

Once a pedicle is cannulated, accessed in any manner or a bone screw is inserted percutaneously, the tube of the facet fusion tube assembly can be clipped onto the associated instrument or tool. In one approach, the tube is passed down along the tool/instrument until the distal end of the tube is positioned directly over the facet capsule and joint, consisting of the superior and inferior articular process of a motion segment. Once the tube is positioned over the facet joint a high-speed burr can be placed down the tube with the centering cap so that the facet joint can be reliably and accurately decorticated and prepared for fusion. Once the facet joint has been decorticated, the tube can then be used to pass morselized allograft, autograft or any FDA approved bone graft material directly into the facet joint and packed in place with the inner rod device passed down the tube.

### BRIEF DESCRIPTION OF THE DRAWINGS

FIG. 1 is an exploded, partial cross-section, view of a facet fusion tube assembly according to the present disclosure.

FIG. 2 is a perspective view of the facet fusion tube assembly shown in FIG. 1.

FIGS. 3A-3D are perspective, end, side and top views of a clip of the facet fusion tube assembly shown in FIG. 1.

FIGS. 4A-4D are perspective, end, side and top views of a centering cap of the facet fusion tube assembly shown in FIG. 1.

FIG. 5 is an exploded, partial cross-section, view of the clip and tube of the facet fusion tube assembly shown in FIG. 1, together with an inner rod disposed within the tube, according to one embodiment of the present disclosure.

FIG. 6 is a perspective view of the clip, tube and inner rod shown in FIG. 5.

FIG. 7 is a side cross-sectional view of the clip, tube and inner rod of FIG. 5, shown in an assembled configuration.

FIG. 8 is a perspective view of the assembled configuration shown in FIG. 7.

FIGS. 9A-9D are perspective, sagittal, coronal and axial views of a lumbar spinal segment with the tube assembly of FIG. 1 aligned over a facet joint and supporting on a tool for accessing the pedicle.

FIGS. 10A-10D are perspective, sagittal, coronal and axial views of the lumbar spinal segment with the tube assembly of FIG. 1 aligned over a facet joint and supported on a bone screw assembly.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

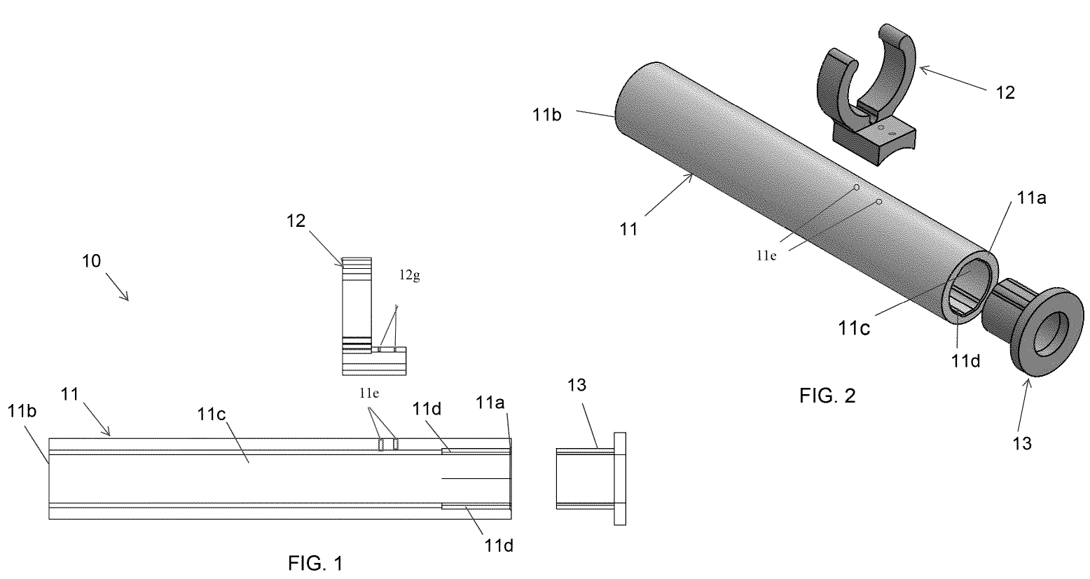

In one aspect of the present disclosure, a facet fusion tube assembly 10 includes a tube 11 and an attachment clip 12, as shown in FIGS. 1-2. The tube 11 is sized for percutaneous introduction into a spinal level with the 11a of the tube accessible outside the patient's body and the distal end 11b adjacent the facet joint at the spinal level. The tube 11 defines a central bore 11e from the proximal to the distal end that is sized to receive instruments and biomaterials for fusion of the facet joint. In one specific embodiment, the tube 11 can have a length between the ends 11a, 11b of 80 mm and an inner diameter of the bore 11e of 10 mm.

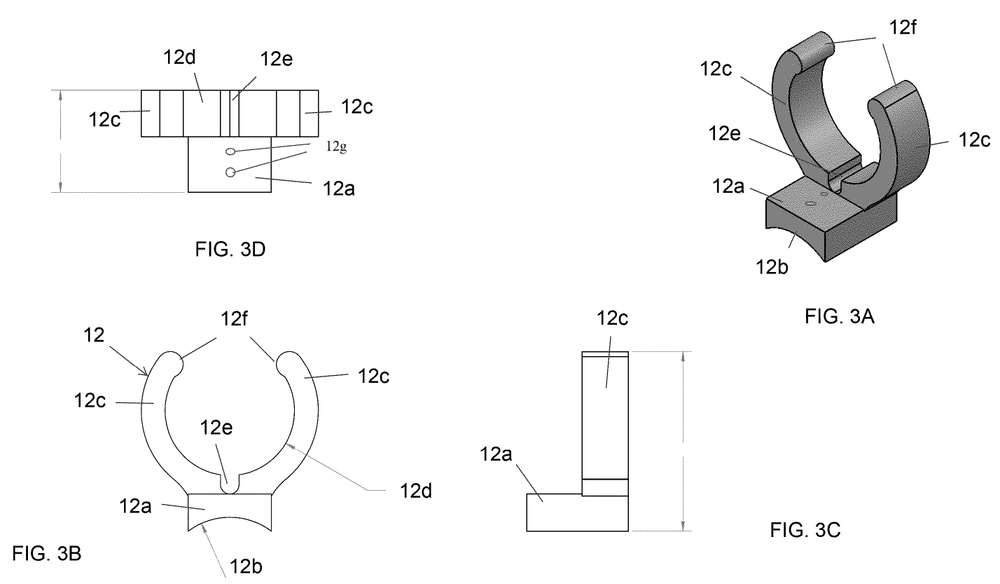

The attachment clip 12, as shown in FIGS. 3A-3D, includes a base 12a with a curved surface 12b sized to conform to the outer diameter of the tube 11. A pair of opposing arms 12e project from the base 12a and define a generally cylindrical interior engagement surface 12d. The arms 12e are configured to flex outward relative to each other to accept a tool or instrument introduced into the engagement surface 12d. A central notch 12e can be provided to facilitate the outward flexing of the arms. A knob 12f can be provided at the free end of the arms to facilitate expansion of the arms as an instrument is introduced into the gap between the knobs. The knobs 12f can also help to retain the tool/instrument within the engagement surface 12d of the clip.

In one embodiment, the base 12a of the clip can have a length of 10 mm, with the arms 12c extending 20 mm from the surface 12b of the base, and thus 20 mm from the surface of the tube 11. The engagement surface 12d can be defined at an effective radius of 7.0 mm so that the clip can clamp onto or engage a tool or instrument having an effective diameter of 14-15 mm. Of course, other dimensions are contemplated that allow the clip 12 to engage the tool or instrument, and particularly that allow the arms 12c to solidly engage the tool/instrument with sufficient force to prevent disengagement of the clip from the tool/instrument.

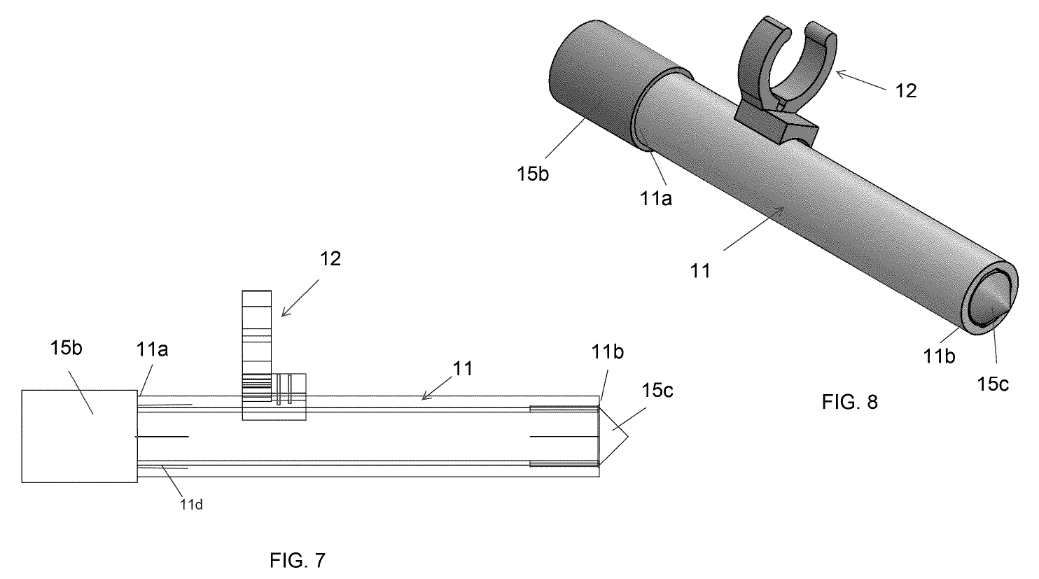

The clip 12 can be attached to the tube 11 at any location along the length of the tube. The attachment is preferably a permanent attachment, such as by welding the base 12a to the tube. Alternatively, the clip 12 can be removably, but rigidly, connected to the tube by passing one or more conventional fasteners through holes 11e and 12g in the tube and the clip base, respectively. In another alternative, the tube can be provided with a series of fastener holes, such as the holes 11e, along the length of the tube to align with the holes 12g in the base 12a to receive the conventional fasteners. The series of holes allow selection of the location of the clip along the length of the tube. Preferably, two sets of holes are provided—one nearer the distal end 11a of the tube, as depicted in FIGS. 7-8, and the other nearer the proximal end 11b of the tube, as depicted in FIGS. 1-2.

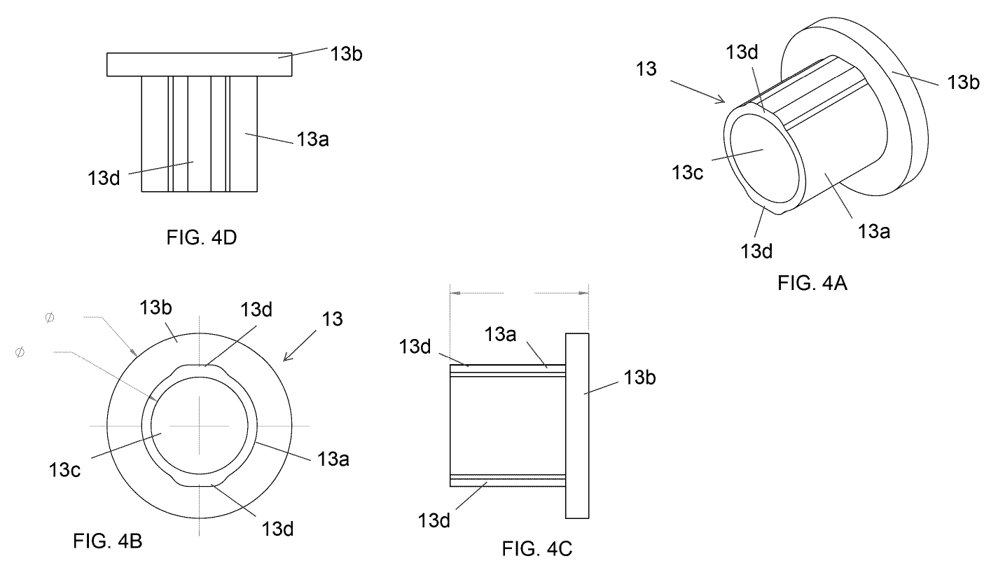

In one embodiment, the tube assembly includes a centering cap 13, shown in FIGS. 4A-4D, that is pressed into the proximal end 11a of the tube (FIGS. 1-2). The cap 13 includes a cylindrical body 13a that is sized for press-fit or friction-fit engagement within the central bore 11c of the tube 11. A plate 13b at the end of the body engages the end of the tube to fix the position of the centering cap within the tube. The body and cap define a centering bore 13e that is sized to accept tools and instruments for performing a facet fusion. To enhance the engagement and to prevent rotation of the cap relative to the tube, the cylindrical body includes a pair of ridges 13d. The proximal end 11a of the bore 11e includes opposing cut-outs or channels 11d that extend a fixed depth into the tube 11 (FIGS. 1-2). The channels 11d are sized and arranged to receive the ridges 13d in nested arrangement when the centering cap is pressed into the tube. In one specific embodiment, the channels 11d are defined at a width between the channels of 4 mm with a depth of 0.67 mm. The cylindrical body 13a of the cap has outer dimensions that substantially match the inner diameter of the central bore 11e and the width of the channels 11d. The plate 13b can have a diameter that is slightly larger than the outer diameter of the tube, such as 14 mm in one specific embodiment. The cylindrical body 13a can have a length of 10 mm, with the depth of the channels 14d being slightly longer than the body of the centering cap. The centering bore 13e has an inner diameter to accept conventional tools and instruments for performing a facet fusion, such as a high-speed burring tool. In a specific embodiment, the inner diameter can be 8.5 mm.

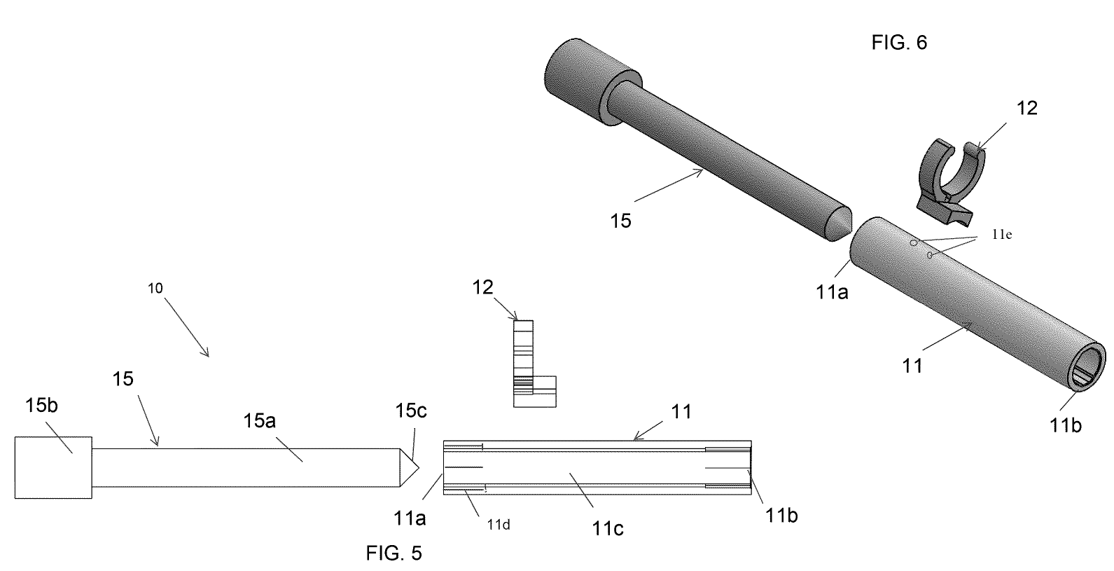

In the illustrated embodiment, a single centering cap 13 is provided at the proximal end 11a of the tube. Alternatively, the centering cap can be provided at the distal end 11b, or a centering cap can be provided at both ends of the tube. In the latter case, channels 11d would be incorporated into the tube 11 at the distal end 11b, as depicted in FIG. 5. However, in the preferred embodiment, the centering cap is only engaged at the proximal end so that the cap can be added to a tube 11 that is already in place within the patient's body, as described herein.

The facet fusion tube assembly 10 can also include an inner rod 15, as shown in FIGS. 5-8. The inner rod includes an elongated shank 15a terminating in an enlarged head 15b at one end and in a conical tip 15c at the opposite end. The shank 15a is sized to extend through the central bore 11c of the tube 11 with the head 15b in contact with the proximal end 11a of the tube and the conical tip 15c projecting beyond the distal end 11b, as best seen in FIGS. 7-8. It can be appreciated that the inner rod 15 is not used when the centering cap 14 is in position. The inner rod has a length from the distal end of the head 15b to the transition from the shank 15a to the conical tip 15c that is at least equal to the length of the tube 11, such as 80 mm in the illustrated embodiment. In this embodiment, the overall length of the rod from end-to-end is 105 mm, and the length of the conical tip is 5 mm. The inner rod 15 is thus sized in relation to the tube 11 so that the conical tip 15c projects beyond the distal end 11b of the tube, as shown in FIGS. 7-8. The enlarged head 14b can have a diameter greater than the outer diameter of the tube, such as 14 mm in a specific embodiment. The elongated shank 15a can have an outer diameter of 8.5 mm, which is slightly less than the 10 mm inner diameter of the bore 11c, thereby providing a close running fit.

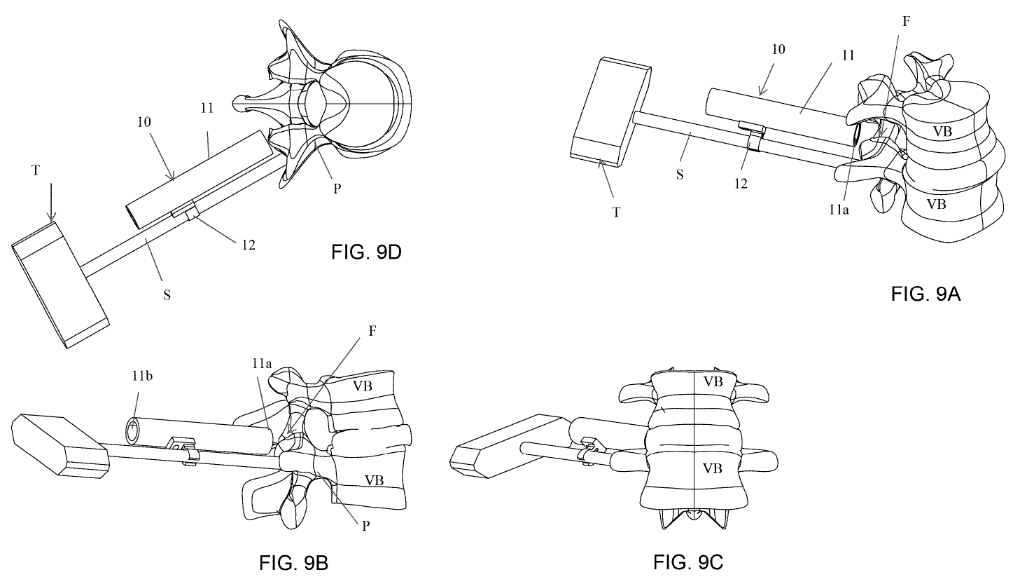

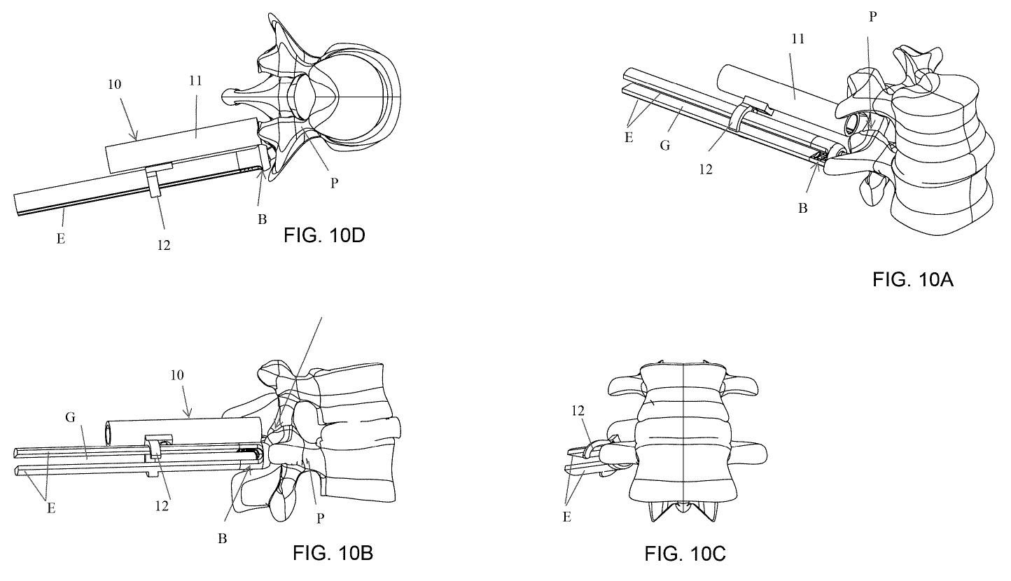

Use of the facet fusion tube assembly 10 of the present disclosure is demonstrated with reference to FIGS. 9A-9D and 10A-10D. Each of these figures depicts a method for accessing the facet joint with the assembly 10 using an adjacent tool or instrument as an anchor element. In FIGS. 9A-9D, the anchor element is a tool T, such as a probe with an elongated shaft S extending into a bore in the pedicle P prepared to receive a bone screw. With the tool T in position, the tube assembly 10 can be introduced into the spine with the inner rod 15 in position, as shown in FIGS. 7-8. The inner rod helps navigate the distal end 11b of the tube through the surrounding soft tissue and prevents any tissue from creeping into the tube. Moreover, the conical tip 15c can dock with the facet joint by contact with the articular processes at the joint. Once the distal end 11b of the tube is properly positioned at the facet joint, the clip 12 can be engaged to the shaft S of the anchor element or tool T. Alternatively, the clip 12 can be engaged to an upper portion of the shaft S, and the clip and tube can slide down the shaft S until the distal end 11a of the tube is properly positioned. With the tube 11 in position, the inner rod can be removed. At this point, the tube 11 is aligned with the facet joint F so that the facet joint can be reliably accessed and prepared for fusion. The tube also provides a cannula for introduction of graft material into the facet joint. The inner rod 15 can be used to push the graft material through the tube 11. The clip 12 is configured to firmly engage the shaft S to hold the position of the tube assembly 10 as the minimally invasive surgical procedure continues.

It is also contemplated that with the tube assembly 10 oriented relative to the facet joint and the inner rod 15 removed, the centering cap 13 can be engaged to the proximal end 11a of the tube 11, in the manner shown in FIGS. 1-2. The centering cap can then be used to guide a working tool, such as a burring tool, used to prepare the facet joint to receive the graft material. The tube assembly 10 can remain engaged to the shaft S of the adjacent tool T during this step of the procedure to ensure that the burring tool is accurately and continuously aligned with the facet joint.

As shown in FIGS. 10A-10D, a bone screw assembly B with frangible extensions E may serve as the guide and anchor element for the tube assembly 10. With the bone screw assembly B engaged in the pedicle P, the extensions E project from the tulip head of the bone screw. The bone screw assembly B is preferably rotated so that the clip 12 spans the gap G between the extensions to ensure that the arms 12c (FIGS. 3A-3D) can adequately grasp the extensions to hold the tube 11 in a fixed position in line with the facet joint F. Once the facet procedure has been completed, the tube assembly 11 can be unclipped from the extensions E and the extensions severed from the tulip head of the bone screw assembly B.

It can be appreciated that the facet fusion tube assembly 10 of the present disclosure, and particularly the clip 12, can be configured to engage any elongated instrument or tool. Thus, rather than the probe T or bone screw assembly B, the tube assembly can be engaged to a working channel cannula that Is anchored to the pedicle P adjacent the desired facet joint F. The clip 12, and particularly the base 12a, is sized so that the tube 11 will be automatically aligned with the facet joint when it is mounted to a tool or instrument that is engaged to the pedicle. The tube 11 will then provide a stable base for performing various steps in the facet fusion process—from preparing the facet joint using a bone burr, for instance, to introducing bone graft material, to packing the graft material into the prepared space using the inner rod.

The components of the facet fusion tube assembly 10 can be fabricated from the same material as the tools and instruments, such as stainless steel. However, since the components are not intended to be load-bearing, they can be formed of suitably rigid polymers. The components can be formed or a radiolucent or non-radiolucent material. The arms 12c of the clip 12 are preferably resilient so that the arms can be deflected outward relative to each other as the clip is engaged on the outer surface of a tool or instrument. The resilient nature of the arms will generate a clamping force that is sufficient to hold the position of the tube assembly 10 on the tool or instrument while the facet fusion procedure is conducted.

What is claimed is:

1. A facet fusion tube assembly comprising:
an anchoring element having an elongated body, with an outer surface, and a distal end configured for engagement in a pedicle of one vertebra of a vertebral level;
a hollow elongated tube having a proximal end, a distal end and a length therebetween that is sized so that when the distal end is positioned immediately adjacent a facet joint of the vertebral level, the proximal end is accessible percutaneously outside the patient, wherein the tube defines a bore having an inner diameter along said length that is sized to accept a tool for preparing the facet joint for fusion; and
a resilient clip fastened to along the length of the elongated tube, the resilient clip having a pair of opposing resilient arms extending outward from said elongated tube and defining a cylindrical interior engagement surface adapted to engage said outer surface of the elongated body of the anchoring element when said hollow elongated tube is laterally adjacent said elongated body of the anchoring element and when said distal end of the elongated tube is positioned immediately adjacent the facet joint, said pair of arms configured to resiliently deflect outward relative to each other for said clip to engage said elongated body of the anchoring element.

2. The facet fusion tube assembly of claim 1, wherein said resilient clip is removably fastenable to an outer surface of said hollow elongated tube.

3. The facet fusion tube assembly of claim 2, wherein:
said resilient clip includes a base; and
said tube and said base define fastener holes in alignment with each other to receive a fastener to removably fasten the clip to the tube.

4. The facet fusion tube assembly of claim 1, wherein said arms define a knob at a free end of each of said arms, said knob configured to slidably engage said outer surface as said clip is engaged to said tube.

5. The facet fusion tube assembly of claim 1, wherein said clip includes a base with said arms projecting from said base, said base defining a notch between said arms to facilitate outward deflection of said arms relative to each other.

6. The facet fusion tube assembly of claim 1, further comprising an inner rod sized to be removably received within the bore of said tube, said inner rod having a conical tip adapted to guide the elongated tube through soft tissue when accessing the facet joint.

7. The facet fusion tube assembly of claim 1, further comprising a centering cap having an elongated cylindrical body sized for a press-fit engagement within said bore, said centering cap defining a centering bore having an inner diameter less than the inner diameter of the bore of said tube.

8. The facet fusion tube assembly of claim 7, wherein the proximal end of said bore of said tube and said cylindrical body define at least one nested ridge and channel interface to prevent relative rotation between said tube and said centering cap.

9. A method for preparing a facet joint at a vertebral level for fusion, comprising:
engaging an elongated anchor element into a vertebral body adjacent the facet joint;
providing a hollow elongated tube having a proximal end and a distal end and a resilient clip fastened along the length of the elongated tube;
extending an inner rod through the elongated tube with a tip of the inner rod extending beyond the distal end of the tube;
introducing the elongated tube and the inner rod into the vertebral level adjacent the anchor element until the tip of the inner rod is in immediate proximity to or in contact with a portion of the facet joint;
engaging the elongated tube to the anchor element using the resilient clip;
removing the inner rod from the elongated tube; and
introducing a tool or instrument into the hollow elongated tube to perform an operation on the facet joint.

10. The method of claim 9, further comprising:
after removing the inner rod, engaging a centering cap to the proximal end of the elongated tube, the centering cap defining a center opening in communication with the hollow elongated tube; and
the step of introducing a tool or instrument includes extending the tool or instrument through the center opening of the centering cap, whereby the centering cap maintains the tool or instrument in a centered position.

11. The method of claim 9, wherein the step of introducing the elongated tube and the inner rod includes;
partially introducing the elongated tube into the patient until the resilient clip is adjacent the anchor element;
then engaging the elongated tube to the anchor element using the resilient clip; and
then sliding the resilient clip along the anchor element to advance the elongated tube and the inner rod into the vertebral level until the tip of the inner rod is in immediate proximity to or in contact with a portion of the facet joint.

12. The method of claim 9, wherein the step of engaging the elongated tube to the anchor element occurs after the tip of the inner rod is in immediate proximity to or in contact with a portion of the facet joint.
